(19) United States
(12) Patent Application Publication
(10) Pub. No.: US 2021/0352176 A1
(43) Pub. Date: Nov. 11, 2021

(54) SYSTEM AND METHOD FOR PERFORMING CONVERSATION-DRIVEN MANAGEMENT OF A CALL

(71) Applicant: KONINKLIJKE PHILIPS N.V., Eindhoven (NL)

(72) Inventors: Wilhelmus Andreas Marinus Arnoldus Maria VAN DEN DUNGEN, Boxtel (NL); Warner Rudolph Theophile TEN KATE, Waalre (NL); Aki Sakari HÄRMÄ, Eindhoven (NL)

(21) Appl. No.: 17/220,049

(22) Filed: Apr. 1, 2021

Related U.S. Application Data
(60) Provisional application No. 63/005,790, filed on Apr. 6, 2020.

(51) Int. Cl. H04M 3/51; H04M 3/493; G16H 50/20; G08B 21/04; G08B 25/01; G08B 29/18; A61B 5/11; A61B 5/16

(52) U.S. Cl. CPC H04M 3/5116; H04M 3/4936; G16H 50/20; A61B 5/165; G08B 25/016; G08B 29/186; A61B 5/1117; G08B 21/043

(57) ABSTRACT

A system for managing a call includes a virtual caregiver that assists callers of a monitoring service in response to data received from a monitoring device. The virtual caregiver includes conversation analyzer that initiates a call to a user of the monitoring device and performs a conversation with the user during the call. The conversation is performed by generating audible comments in a synthesized voice to interact with the user. The audible comments are generated to elicit voice responses from the user containing information corresponding to the sensor data. The conversation analyzer also analyzes audible features of the voice responses using one or more models to interpret a condition of the user, generates a decision based on the interpreted condition of the user, and performs at least one action based on the decision.

### CROSS-REFERENCE TO PRIOR APPLICATIONS

This application claims the benefit of U.S. Provisional Application No. 63/005790, filed on 6 Apr. 2020. This application is hereby incorporated by reference herein.

### TECHNICAL FIELD

This disclosure generally relates processing information, and more particularly to a system and method for performing conversation-driven management of a call.

### BACKGROUND

A variety of devices have been developed to monitor and protect the safety of elderly patients. Examples include cameras, medical alert systems, trackers, and fall detectors. Fall detectors have proven to be especially popular as a first-responder system. For example, when a fall occurs and an alarm is triggered, a call center service may contact the user to investigate. If this service is able to determine that the user is in an emergency situation, additional care resources may be dispatched.

Because of their high rate of false alarms (e.g., once per day), many fall detectors have a cancel button. In order to be effective, the cancel button must be used within a predefined or adaptive waiting period after the alarm has been triggered. Knowing this, many call centers delay action until after expiration of the waiting period, in case the alarm is a false alarm. Such a waiting period may be detrimental to a user when the alarm is for an actual fall.

For many elderly persons, the cancel button may prove to be somewhat of a technical challenge. This challenge may be exacerbated when the fall detector includes additional functional controls, such as a help or alarm button. For persons with dementia or other cognitive disease, having to find and then figure out the right button to push may cause confusion and diminish the efficacy of the device for purposes of obtaining help when needed and/or canceling false alarms to prevent a waste of valuable emergency resources.

In some cases, the cancel or help button may be misused. For example, an elderly person may cancel an alarm when he has fallen, either because of embarrassment or out of fear that a loved one or caregiver may recommend relocation to an assisted living facility or nursing home. Existing call center services that are software-driven are unable to determine whether the user is in distress even though the cancel button was pushed. In other cases, the help button may be pressed by an elderly person who is lonely and just wants to talk with someone.

Call centers are also unable to determine whether the user has actually fallen. Moreover, once contact has been made, call center software is unable to distinguish whether the user is in denial of an actual fall or is in a confused or other ambiguous state. Call center software is also unable to learn from earlier conversations with the same user or ones with other users who may be similarly situated.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In one embodiment, a system for managing a call includes a memory configured to store instructions; and a processor configured to execute the instructions to implement a virtual caregiver to assist callers of a monitoring service, the virtual caregiver configured to receive sensor data from a monitoring device and activate a conversation analyzer in response to the sensor data, the conversation analyzer configured to: initiate a call to a user of the monitoring device; perform a conversation with the user during the call, the conversation performed in accordance with operations that include generating audible comments in a synthesized voice to interact with the user, the audible comments to elicit voice responses from the user containing information corresponding to the sensor data; analyzing, using one or more models, audible features of the voice responses to interpret a condition of the user; generating a decision based on the interpreted condition of the user; and performing at least one action based on the decision.

The audible features may include one or more of voice inflection, pitch pattern, tone variations, volume fluctuations, or variable speech patterns. The condition may be one of an intent, emotional state, or mental state of the user. The one or more models may be artificial intelligence models that are to be trained based on patterns of audible features personalized to user, and the processor may update training of the one or more models based on at least one of the voice responses, decision, or interpreted condition of the user.

The monitoring device includes a fall detector and the sensor data may indicate that the user has experienced a fall. The conversation analyzer may generate a score based on output of the one or more models, the score indicative of a probability of the decision based on the interpreted condition. The interpreted condition may include an actual fall of the user; the decision may be the user is denying that the actual fall occurred, and the at least one action may include generating signals to perform one or more of passing the call to a live operator, notifying an emergency resource for the user, or notifying a live caregiver.

The interpreted condition may include that the user did not actually fall, the decision may be the user has pushed an alarm button on the fall detector in order to have a social call, and the at least one action may include generating signals to perform one or more of activate an artificial intelligence bot to generate dialog that provides options to the user being monitored during a conversation or obtain additional information or notifying a live caregiver. The interpreted condition may include the user is in a confused state, the decision may be the user requires assistance, and the at least one action may include one or more of notifying an emergency resource for the user or notifying a live caregiver. The conversation analyzer may generate the decision based on the interpreted condition of the user and one or more of information included in the sensor data or profile information of the user.

In accordance with one or more embodiments, a method for managing a call includes receiving sensor data from a monitoring device and activating a conversation analyzer in response to the sensor data, wherein activating the conversation analyzer includes initiating a call to a user of the monitoring device; performing a conversation with the user during the call, said performing including generating audible comments in a synthesized voice to interact with the user, the audible comments eliciting voice responses from the user containing information corresponding to the sensor data; analyzing, using one or more models, audible features of the voice responses to interpret a condition of the user; generating a decision based on the interpreted condition of the user; and performing at least one action based on the decision.

The audible features may include one or more of voice inflection, pitch pattern, tone variations, volume fluctuations, or variable speech patterns. The condition may be one of an intent, emotional state, or mental state of the user. The one or more models may be artificial intelligence models that are trained based on patterns of audible features personalized to user, and the method may include updating training of the one or more models based on at least one of the voice responses, decision, or interpreted condition of the user. The monitoring device may include a fall detector and the sensor data may indicate that the user has experienced a fall.

The method may include generating a score based on output of the one or more models, wherein the score is indicative of a probability of the decision based on the interpreted condition. The interpreted condition may include an actual fall of the user; the decision may be the user is denying that the actual fall occurred; and the at least one action may include generating signals to perform one or more of passing the call to a live operator, notifying an emergency resource for the user, or notifying a live caregiver.

The interpreted condition may include that the user did not actually fall, the decision may be the user has pushed an alarm button on the fall detector in order to have a social call, and the at least one action may include generating signals to perform one or more of activate an artificial intelligence bot to generate dialog that provides options to the user being monitored during a conversation or obtain additional information or notifying a live caregiver. The interpreted condition may include the user is in a confused state, the decision may be the user requires assistance, and the at least one action may include one or more of notifying an emergency resource for the user or notifying a live caregiver. Generating the decision may be performed based on the interpreted condition of the user and one or more of information included in the sensor data or profile information of the user.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a system including a virtual caregiver for managing a call;

FIG. 2 illustrates an embodiment of a conversation analyzer;

FIG. 3 illustrates an embodiment of logic of the conversation analyzer;

FIG. 4 illustrates an embodiment of a method for implementing a virtual caregiver for managing a call; and

FIGS. 5A to 5C illustrate example applications of the system and method.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

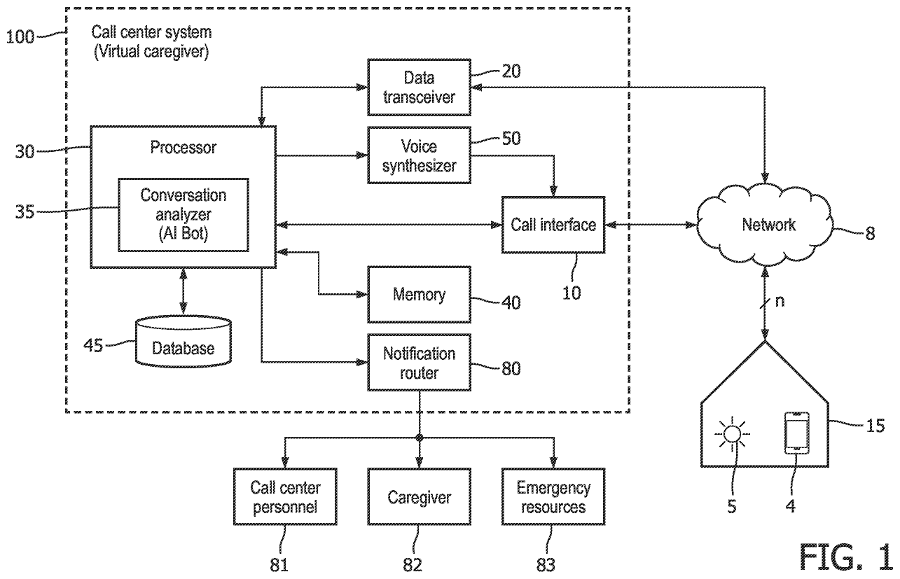

FIG. 1 illustrates a system 100 for performing conversation-driven management of a call initiated based on data generated by one or more sensors. The system may be implemented, for example, at a call center, an emergency dispatch center (e.g., 911 or other service), a monitoring service, or another location that accepts calls for rendering public or private services to persons associated with the one or more sensors. For illustrative purposes, the system is depicted as a call center system implementing a virtual caregiver based on stored instructions, which virtual caregiver includes a conversation analyzer which operates to manage a call in response to the data from the one or more sensors.

Referring to FIG. 1, the system includes a call interface 10, a data transceiver 20, a processor 30, a memory 40, and a voice synthesizer 50. The call interface 10 receives calls from various persons, including ones being monitored by the one or more sensors. The call interface receives calls from and/or initiates calls through a mobile communications system, a landline, the internet, or another voice or data communication system in the form of voice, video, or both. The communication system(s) may generally be referred to as network 8. In one embodiment, a call may be initiated by call center system in response to data received from the one or more sensors. The call may be directed to a person being monitored by the one or more sensors. The person, for example, may be in his house, a care facility (e.g., assisted living, nursing home, etc.), or another location 15. In other embodiments, one or more features at the call center may be implemented locally, for example, at the monitoring location.

The one or more sensors 5 may be used to monitor various types of information. For example, at least one of the sensors may monitor the location of the person, either locally throughout the house or at indoor and outdoor locations. Another sensor may track the motion or movement of the user at these locations. Another sensor may monitor the health condition of the person. An example is a medical alert device which monitors vital signs (e.g., heart rate, blood pressure, etc.) of the person and then sends a notification, on a periodic basis and/or when an anomaly is detected. Another sensor may be a camera. Another sensor may be a fall detector. A fall-detector embodiment will be discussed in greater detail below, with the understanding that any one or more of the aforementioned types of sensors may be used with the call center system described herein. In one or more embodiments, different types of monitoring or alarm systems (e.g., other than or in addition to fall detector alarms) may be used for conversation-driven call management.

In addition to sensor(s) 5, the person being monitored has access to a phone or other communication device including audio functionality capable of making calls that are received by the call interface. Examples of the device include a smartphone, landline phone, or voice-over-internet device or application. In one embodiment, the sensor(s) and devices may communicate with the call center system over different networks, e.g., a data network and a mobile communications network. To show this, the connection between the house and network 8 is showing as being comprised of n types of communication links, where n≥1.

The data transceiver 20 receives the data transmitted from the one or more sensors monitoring the person at house 15. In one embodiment, the data receiver may be a personal emergency response system (PERS) transceiver which receives signals from the sensor(s) 5 (e.g., a Philips Lifeline device) when the person is in potentially exigent or other circumstances. In another embodiment, the data transceiver 20 may be another type of communication device for receiving sensor data different from a PERS transceiver. The signals received from sensor(s) 5 may take a variety of forms. For example, the signals may include a passive alarm indicating generally that the person is in some kind of trouble or otherwise requires assistance. In another embodiment, the signals may contain more substantive information, such as vital signs or other medical information. In the case of a fall detector, the signals may include a notification that the person has fallen and that a response from the call center system may be required in order to investigate. In one embodiment, investigation of a fall or other condition of the person may be performed with various different emergency triggers. For example, when a trigger occurs, a procedure may be followed to determine whether the received alarm is a false alarm. The procedure may involve an automated (e.g., software-driven) call manager (or call center or e911 operator) attempting to contact the person and/or send confirmation signals to the monitoring device that generated the alarm in order to verify the status of the alarm, and also to gain more insight into what actually may be going on in the real situation.

The processor 30 may control the operations of the virtual caregiver of the call center system. These operations may be performed automatically in accordance with instructions stored in memory 40 or interactively based on instructions stored in memory 40 and/or manual input from one or more call center personnel. In operation, when data is received from sensor(s) 5, the processor is triggered to automatically initiate a call to phone of the person being monitored in house 15. The call may not be made by a person but may be performed based on logic determined by an interactive program that generates dialog with the person in order to engage in a conversation. The logic may include or implement an artificial intelligence (AI) bot.

In one embodiment, the AI bot may correspond to a computer program, executed by processor 30, that generates dialog for the purpose of eliciting responses and holding a conversation with a person being monitored. The dialog generated during the conversation is based on artificial intelligence models, for example, as described herein. Based on these models, the AI bot may generate person-specific questions and may perform person-specific analysis of information gleaned during the conversation. For example, in one case, the dialog generated by the AI bot may be different when the same answer is given by multiple persons being monitored. The different dialog is a function of the artificial intelligence models, which may be specifically customized to the person through machine-leaning techniques and/or other considerations used to train/design the model(s).

The voice synthesizer 50 generates artificial speech directed at gaining specific information from the person during the conversation. For example, under programmed control of the processor (e.g., using the AI bot), the voice synthesizer may ask questions such as did a fall occur, how did the fall occur, when did the fall occur, are you hurt, do you need emergency services, etc. These operations cause therefore cause the processor to operate as a Virtual Caregiver for the person, which may alone handle the call or take other action, for example, after certain information is gathered. The answers to the questions, voice characteristics, and/or sensor data may be analyzed. In one embodiment, an iterative process (e.g., such as a 5-Times-Why methodology) may be initiated based on signals from a personal emergency response system (PERS) and/or E911 protocols. Such an iterative process may involve looping questions and answers in order to receive additional information that may be useful in formulating a decision on any of the scenarios described herein.

The responses to the questions are then analyzed to make one or more determinations. Examples of these determinations include (1) whether a fall actually occurred but the person is denying that it happened, (2) the person is in a confused state either from the fall or from another condition, (3) the circumstances that caused the sensor(s) to transmit the data to the call center system are complicated to understand and/or the person is having a difficult time explaining those circumstances, or (4) whether a real emergency exists or whether the person (e.g., elderly) is just lonely or wanting some social interaction and feigning that a fall occurred. The first situation (1) may occur, for example, when the sensor device includes a cancel alarm button that is manually pushed by the person being monitored. The latter situation (4) may occur, for example, when the sensor (e.g., fall detector) 5 includes an alarm button that allows the person to manually generate an alarm. Additional determinations or decisions may be made based on further analysis of the conversation.

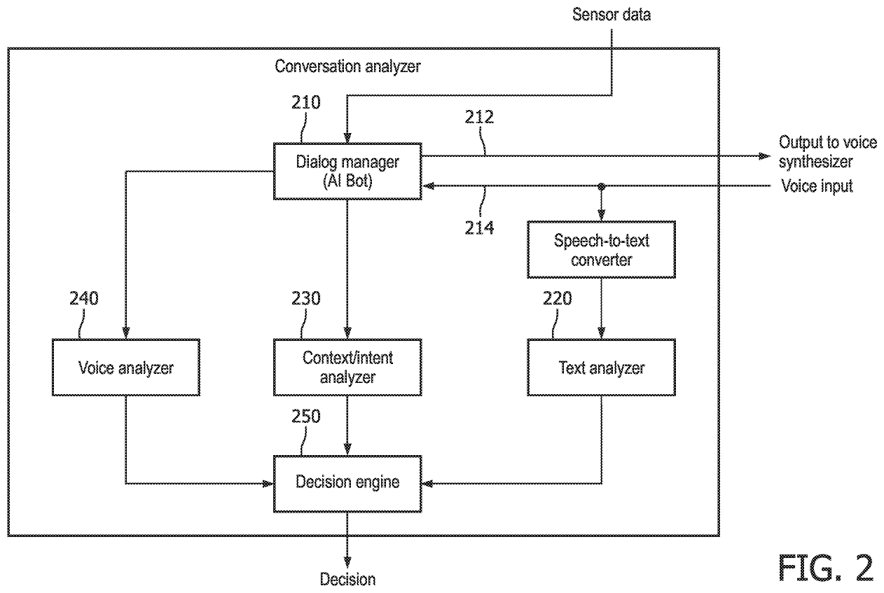

FIG. 2 illustrates an embodiment of a conversation analyzer 35 which may be implemented by the processor 30 to perform the conversation analysis. In this embodiment, the conversation analyzer includes a dialog manager 210, a text analyzer 220, a context/intent analyzer 230, and a voice analyzer 240. Additional sensors and event information may be used, along with alarm rate, fall detection confidence, and other considerations, data, and parameters.

The dialog manager 210 controls the operations for analyzing the conversation between the person being monitored and the call center system, which in this case is operating as a virtual caregiver. The operations include interactively managing the conversation after the processor 30 initiates a call to the phone (or other communication device) 4 of the monitored person, which initiation may be performed in response to an alert or other sensor data received by the data transceiver. After the person answers the call, the dialog manager (e.g., using the AI bot) generates information (e.g., dialog) 212 which is output to the voice synthesizer. The voice synthesizer converts this information into an audible introductory greeting which serves as the virtual voice of the call center system. The virtual voice passes through the call interface and the network in order to reach the phone 4 (or other audio device/method) of the person being monitored. The introductory greeting may, for example, identify the virtual caregiver and the reason for the call, e.g., “Hello, this is Philips Lifeline calling. We received a notification that you may have fallen. Are you alright ?”

Once the person responds to the introductory greeting, the dialog manager 210 may receive the voice response 214 from the call manager. The voice response from person is then analyzed using the processing logic of the conversation manager 35. Based on the results of the analysis, the dialog manager generates questions, comments, and/or other information that is synthesized into the voice of the virtual caregiver. The conversation then ensues, with each response being analyzed by the processing logic of the conversation manager to make various determinations and assessments, for example, as previously described.

The processing logic of the conversation manager 35 may vary among embodiments. In one embodiment, the processing logic includes the text analyzer 220, the context/intent analyzer 230, and the voice analyzer 240. The text analyzer 220 is coupled to a speech-to-text converter 215, which converts the vocal responses of the monitored person during the call into text. The text is then analyzed for content by the text analyzer. For example, the text may be analyzed by performing a keyword search. Certain keywords may be recognized by the text analyzer as corresponding to certain actions. For example, a response indicating that “Nothing is wrong” may be immediately flagged by the text analyzer as warranting further investigation as to whether a fall actually occurred, and the monitored person is in denial. In such a case, the output of the voice analyzer or the context/intent analyzer, or both, may be considered in arriving at a decision as to whether the person is in denial of an actual fall. In these or other cases, models and or algorithms may be implemented to recognize keywords specific or personally relating to the person being monitored. These algorithms or models may, for example, be trained to perform this level of recognition.

In another case, responses that are interpreted as not being indicative of an emergency, but rather on a more social level, may warrant a decision that perhaps the monitored person is lonely and pressed an alarm button on the alert device just to talk with someone. In this case, information in the person's profile (e.g., stored in database 45) indicating that the person is elderly and lives alone may help in arriving at this decision.

The context/intent analyzer 230 may analyze the environmental and contextual data. For example, an event occurring at night time may have a different context than a daytime event. In one embodiment, the analyzer may be a classifier possibly based on various features related to the time, environmental conditions, sensor data and/or the output of the speech and text analyzer. The C/I analyzer has been trained to recognize a set of predefined context/intent settings. Typical examples are “daytime activity”, “night time event”, “heatwave”, “visitor with the subscriber”. In addition, the context/intent analyzer may constantly be updated/trained based on the event annotated conversations where the algorithm “Listens in” and is personalized and trained based on the specific subscriber's behavior/intent.

The voice analyzer 240 may perform a spectrum analysis on the voice responses received by the monitored person to assess, for example, voice inflection, excitability, tone, and other indicators that may for a basis for better interpreting the content of the responses. In one embodiment, the results of the spectrum analysis may be combined, for example, with other information such as keywords and history responses in order to generate results with improved accuracy.

A decision engine 250 generates a decision, taking into consideration the outputs of the text analyzer, the context/intent analyzer, the voice analyzer, relevant sensor data (e.g., vital signs, parameters, etc.), and/or information stored in the database profile of the person being monitored. Such an engine may be implemented, for example, as a classifier which is pre-programmed with a knowledge base and/or machine-learning algorithm to classify calls based on the aforementioned outputs. The decision generated by the decision engine 250 may be used by the processor as a basis for controlling the disposition of the call. For example, as illustrated in FIG. 1, the processor may generate control signals for performing various actions based on the decision. The control signals may be sent to a notification router 80, which passes control to one or more of a live operator 81 at the call center, an emergency contact 82 (e.g., caregiver, relative, guardian, etc.), or emergency resources 83 (e.g., fire department, paramedics, etc.).

In one embodiment, the decision may be expressed as a probability or likelihood of a computed determination. For example, the decision engine 250 includes logic to generate a score based on the outputs of the text analyzer, the context/intent analyzer, the voice analyzer, relevant sensor data (e.g., vital signs, parameters, etc.), and/or information stored in the database profile of the person being monitored. The processor 30 may then generate control signals for performing various actions based on the score. For example, the process may compare the score to one or more predetermined ranges, where each range determines the action to be take.

Table 1 shows an example of actions that may be taken for various ranges of scores generated when a fall detector has transmitted an alarm or other form of data to the call center system and the conversation analyzer 35 generates a decision based on the information described herein.

| Decision | Probability Score | Actions |
|---|---|---|
| Denial of Actual Fall | First Range (85-100) | Dispatch Emergency Resources |
| and/or Emergency | | and Pass to Call Center Operator |
| | Second Range (60-85) | Notify Caregiver and Pass to Call |
| | | Center Operator |
| | Third Range (<60) | Pass to Virtual Caregiver AI Bot |
| No Actual Fall and/or | First Range (80-100) | Pass to Virtual Caregiver AI Bot |
| Emergency | Second Range (<80) | Pass to Call Center Operator |
| Confused State, Not Sure | First Range (70-100) | Notify Caregiver and Pass to |
| of Fall Status and/or | | Call Center Operator |
| Emergency | Second Range (<70) | Notify Caregiver and Pass to |
| | | Virtual Caregiver AI Bot |

The first type of decision is denial of an actual fall. This may occur, for example, when the fall detector has detected that the person being monitored has fallen and then automatically sends an alarm signal to the call center system. The alarm signal may be simply be a signal indicating an alarm or may include more substantive information generated by the fall detector and/or one or more other sensors at the monitored person's location. For example, the more substantive information may include the location in the home where the fall occurred, the person's vital signs, etc.

The denial of the actual fall may also be based, for example, on additional information received from the fall detector. For example, when the fall detector includes a cancel button, the additional information may be receipt of a cancel signal received at the call center. In another case, the denial may result based on the information input into the conversational analyzer, including the voice responses given by the monitored person during the conversation with the virtual caregiver.

The scores generated by the conversational analyzer 35 may, in this example, fall into one of three predetermined ranges indicative of the severity or probability that the monitored person is denying that an actual fall has taken place. The first range covers scores of 85 to 100, indicating that there is a high probability that an actual fall has taken place which is being denied. In this case, the processor 30 may generate control signals for the notification router to dispatch emergency resources and pass the call to a live operator at the call center.

The second range covers scores of 60 to 85, indicating that there is a moderate probability of an actual fall being denied. In this case, the processor 30 may generate control signals for the notification router to contact a caregiver and pass the call to a live operator at the call center. The caregiver name and contact information may be retrieved from profile data stored, for example, in database 45.

The third covers scores of less than 60, indicating that there is a low probability that the monitored person is denying an actual fall. In this case, the processor 30 may generate control signals for activating a virtual caregiver AI bot to generate dialog (including but not limited to additional questions) for the monitored person in order to gather more information, so that a more definitive decision may be made or so that other preprogrammed actions may be taken based on the additional information.

The scores generated by the conversational analyzer 35 may fall into one of two predetermined ranges indicative of the severity or probability that no fall has actually taken place, even though data has been received from the fall detector indicating a fall. This may occur, for example, when the fall detector has issued a false alarm or when the alarm button has been pushed on the false detector and the monitored person is lonely and just wants to talk with someone or wants to socialize. A call in this category may be classified as a social call.

The first range covers scores of 80 to 100, indicating that there is a high probability that no fall has actually taken place even though the fall detector has transmitted data indicating otherwise. In this case, the processor 30 may generate control signals for controlling an AI bot of a virtual caregiver to generate additional dialog with the person being monitored in order to continue the conversation in attempt to determine more information.

The second range covers scores of less than 80, indicating that there is a lower probability of a false indication of an actual fall. In this case, the processor 30 may generate control signals for the notification router to pass the call to a live operator at the call center, in order to provide an opportunity for further investigation as to the indicated fall and the general health and status of the person being monitored.

The scores generated by the conversational analyzer 35 may fall into one of two predetermined ranges indicative of the severity or probability that the monitored person is in a confused state and the status of the fall is unclear. That may occur, for example, when data is received from the fall detector indicating that a fall has taken place and the voice responses from the monitored person do not make sense, are unintelligible, or otherwise deviate from expected responses. In this case, the monitored person may have sustained a concussion from the fall and is unable to articulate clear responses to the questions of the virtual caregiver. This may also occur when the monitored person is suffering a stroke, has dementia, or is undergoing a seizure or some other medical condition that requires attention.

The first range covers scores of 70 to 100, indicating that there is a high probability that the monitored person has suffered a fall and has a concussion or is experiencing a serious medical episode requiring attention. In this case, the processor 30 may generate control signals for the notification router to notify the designated caregiver and connect the call to a live operator at the call center.

The second range covers scores of less than 70, indicating that there is a lower probability that the monitored person has suffered a fall (e.g., may have pushed the cancel or alarm button) or is in a state that requires immediate attention. Because the circumstances are unclear, further investigation is warranted. Thus, the processor 30 may generate control signals for the notification router to contact the designated caregiver and then control an AI bot of a virtual caregiver to generate additional dialog with the person being monitored in order to continue the conversation in attempt to determine more information.

This multi-tiered scoring approach allows the conversation analyzer 35 to implement a virtual caregiver service that operates as a filter, to determine whether an actual fall has occurred and prevent connecting the monitored person to a live operator, emergency services and/or the use of other valuable resources in cases which are less likely to require those resources. This may advantageously free up those resources for more serious cases where they are actually required, while at the same time being attentive to the needs of the monitored person through administration of a virtual caregiver service driven by the conversation analyzer.

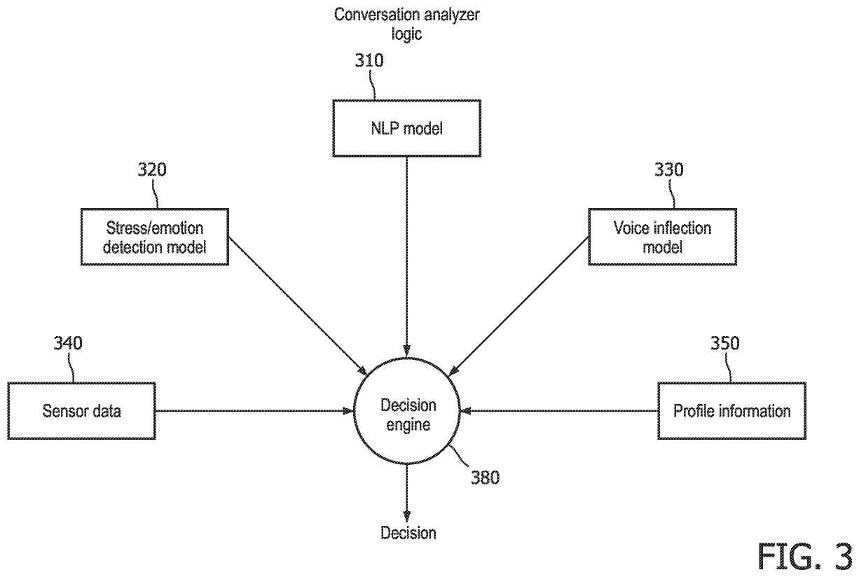

FIG. 3 illustrates an embodiment of the logic of the conversation analyzer 35. This logic includes a natural language processing (NLP) model 310, a stress and/or emotion model 320, and a voice inflection model 330. The outputs of these models may be input into the decision engine 380, which generates a decision for managing an event corresponding to the sensor data received by the call center system. The decision may indicate a yes or no answer or may involve generating a probability score as previously described. In one embodiment, the decision engine may generate the decision based on fewer than all three models (e.g., any one or two of the models). The decision may also be generated with reference to information in the sensor data and/or profile information stored by the system for the person being monitored.

Referring to FIG. 3, the NLP model 310 may process the incoming voice signal of the monitored person by performing one or more of syntax analysis, semantics analysis, and discourse analysis. Syntax analysis may involve, for example, performing grammar induction, lemmatization, morphological segmentation, part-of-speech tagging, parsing, sentence braking, stemming, word segmentation, and/or terminology extraction of the received voice signal. The semantics analysis may involve, for example, performing lexical semantics, distributional semantics analysis, language translation, named entity recognition (NER) analysis, relationship extraction, natural language understanding techniques, and/or disambiguation. Discourse analysis may involve automatic summarization, coreference resolution, and an analysis of speech discourse relationships.

The stress and/or emotional model 320 may analyze the voice signals received from the monitored person during a conversation to detect indications of stress or emotion. The stress/emotional model may be different from the voice inflection model in a number of ways. For example, the inflection may also be used to detect different grammatical intent and meanings of words. In one or more embodiments, voice inflection may refer to the modification of a word to express different grammatical categories, such as tense, grammatical mood, grammatical voice, aspect, person, number, gender and case. Conjugation may include the inflection of verbs, and declension may include the inflection of nouns, adjectives and pronouns. In other embodiments, the stress/emotional model may correspond to the voice analyzer or the context/intent analyzer of FIG. 2.

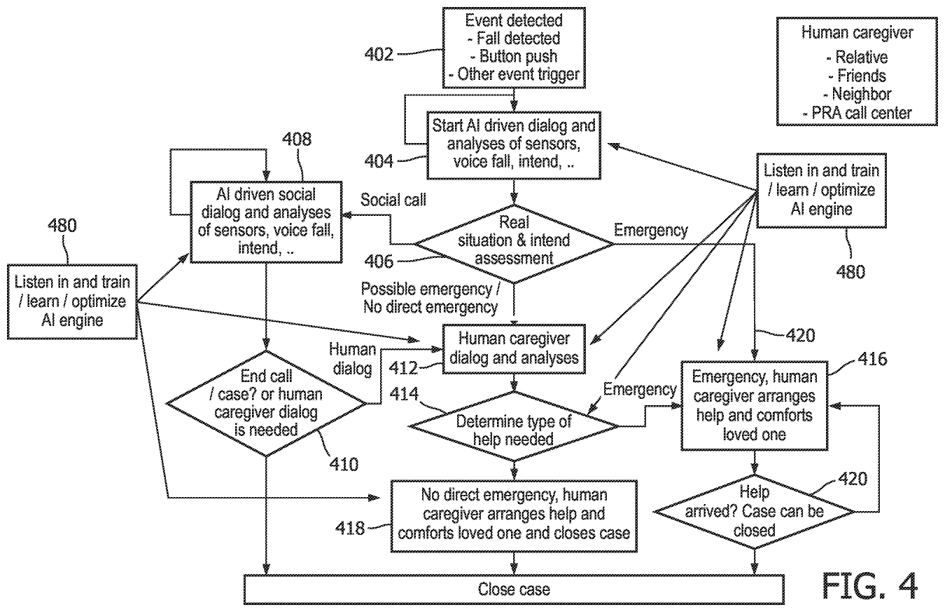

The voice inflection model 330 may analyze the voice of the monitored person to, for example, determine voice pitch patterns, volume, and tone. This model may also determine how fast or slow the person is talking, whether the voice is a shaky or has unstable or variable speech patterns, whether the person is stuttering or stammering, or whether the person is crying, laughing, shouting, or exhibiting some other form of emotion. In one embodiment, audio of the utterances the monitored person made during the alleged fall may be recorded, for example, by a sensor (e.g., smartphone microphone or other sound-capturing detector) at the scene. These utterances may be analyzed, for example, to determine the authenticity of the fall. All of this information may be considered to be initialization information. During use, the model may be updated based on learning and new keywords used during events, voice inflections, emotions, sensor readings in order to provide a personalized model. In one embodiment, cross-user learning may be performed in order to update the baseline. See, for example, the features of FIG. 4.

All of this information may be compared to reference patterns to ascertain the intent or mental state of the monitored person. This information may be used as a basis for determining, for example, whether the person is in denial of an actual fall or whether the call is actually a social call disguised as a distress call, for example, through activation of the alarm button on the fall detector. In one embodiment, the voice inflection model may correspond to the voice analyzer or the content/intent analyzer of FIG. 2.

In one embodiment, the models 310, 320, and 330 may be implemented using artificial intelligence to provide a more accurate analysis of the voice signals of the monitored person during a call. One example of an artificial intelligence application is a machine-learning algorithm, neural network, or other model-based logic which is trained based on personal data that relates to the person being monitored. The training data may initially involve taking voice samples of speech patterns, inflections, speech traits, and other verbal behavior characteristics and idiosyncrasies of the monitored person. This information may provide a baseline or reference for how a person normally talks, which may be contrasted to the voice of the person during calls to provide an indication of emotion, stress, intent, and/or other properties relevant to generating a decision by the decision engine. The processor 30 may then update the training data of the model during subsequent calls, in order to allow the model to learn the specific nuances relating to the person being monitored. This learning process produces a model which is more adept at generating an accurate analysis of the specific person, which, in turn, may produce a decision that can predict exactly what type of care and service the person needs when the fall detector is triggered. The training data and data obtained for each call may be stored in the database for access by the conversation analyzer for performing the operations described herein.

The sensor data 340 may include data from any of the types of sensors described herein. For example, the sensor data may include accelerometer data that may provide an indication of how severe an actual fall might have been given measured acceleration forces. The sensor data may also include images or video taken from a camera in the house of the person being monitored. These images or video may serve as additional information which may allow the decision engine to infer what actually happened in order to trigger the data received from the fall detector. Other types of sensor data may also be taken into consideration by the decision engine when generating a decision.

The profile information 350 may include various types of personal information for the person being monitored. This information includes demographics such as age, sex, address, etc., medical history records including relevant diseases or conditions the person may have, prescriptions data indicating the medicines currently being taken and that were taken in the past but are no longer being used, indications of limitations on mobility, and other information that may provide a basis for assessing health. The profile information may also include emergency contact and caregiver information, health insurance information, and event history information for multiple events occurring over a previous time period, e.g., day, week, etc. The more information considered, the more pattern features may be determined and the greater the stability.

The decision engine 380 may generate the decision concerning how the call is to be managed based on one or a combination of outputs of the models, sensor data, and profile information. In one embodiment, the decision engine 380 may generate the decision score based on Equation 1.

Decision Score=α₁·O₁+α₂·O₂+α₃·O₃+α₄C₁+α₅C₂ (1)

In Equation 1, α₁, α₂, and α₃ are weights assigned by an algorithm of the decision model to the importance of the outputs generated by individual ones of the models 310, 320, and 330. In one embodiment, the weights may be determined using a machine-learning algorithm, such as but not limited to logistic regression. The values C₁ and C₂ are values assigned by the decision engine that are weighted by respective coefficients α₄ and α₅, based on the importance of information in the sensor data and profile, respectively, to the decision. In one embodiment, the sensor data and/or profile information may be input into respective ones of models 310 to 330 in order to generate their respective outputs.

FIG. 4 illustrates a method for performing conversation-driven management of a call initiated based on data generated by one or more sensors. The method may be performed, for example, by the system, models, and other logic of FIGS. 1 to 3 or may be performed by a different system, set of models, or logic.

Referring to FIG. 4, at 402, the call center system detects an event has occurred by receiving a signal from the fall detector of a person being monitored. The signal may be an alarm signal and/or may include sensor data output from the fall detector and/or a suite of other sensors in operation at the monitoring location. The received signal may be generated when an event trigger occurs, e.g., when an accelerometer in the fall detector senses forces that indicate that the wearer has fallen. In one or more embodiments, the event trigger may be derived from various types of event sensors, heart rate sensors, smoke detection sensors and blood pressor sensors, as well as others. In some cases, the signal may be generated when an alarm button on the fall detector is pushed. In other cases, a second signal may be received for cancelling the initially received fall detection signal.

At 404, the processor receives the sensor data through the data transceiver and activates the processor 30 to initiate a call through the call interface. When the monitored person (or an automated answer system) answers the call, the processor activates the conversation analyzer 35 and an initial greeting is generated and output through the voice synthesizer to begin the conversation with the virtual caregiver application implemented by the call center system. The voice responses are than analyzed, for example, using one or more of the artificial intelligence models previously discussed (e.g., see FIG. 3).

At 406, the decision engine of the conversation analyzer generates a decision based on the outputs of the models and the sensor data and profile information. The decision may, for example, be one of the decisions indicated in Table 1. In one embodiment, the decision may be generated as a score, for example, based on Equation (1) or using another formula or algorithm.

At 408, when the decision indicates that the voice responses of the person being monitored is merely a social call (e.g., no actual fall has occurred), the conversation analyzer may pass control of the call to an AI bot of a virtual caregiver to generate additional dialog with the user being monitored in order to continue the conversation in attempt to determine more information and/or to provide the user with options. In some cases, depending on the score, a caregiver or other entity may be contacted.

At 410, after the conversation between the AI bot and the user is completed, the call may be ended. In one embodiment, if the additional information received from the monitored person in response to the dialog generated by the AI bot changes the decision of the call, then the processor 30 pass the call on to a live operator at the call center or a caregiver may be contacted to visit or help the person. In one embodiment, the voice responses generated during the conversation initiated by the AI bot may be routed back through the conversation analyzer models to generate a revised decision and its attendant action.

At 412, when the decision indicates that there is a possible emergency or there is no direct emergency (e.g., as indicated by a corresponding score), then the processor may take the corresponding action indicated in Table 1. For example, this may involve passing the call to a human caregiver, who may then conduct a follow-up conversation with the person, and/or an AI bot may be activated. Such a decision may occur, for example, when the monitored person is determined to be in a confused state based on his voice responses or the fall status is otherwise unclear.

At 414, if the call is passed to a human caregiver, then the caregiver may determine the appropriate action to take. For example, when the caregiver determines that the monitored person is in a severe state, either because of an actual fall, because of a stroke or other health condition, then the human caregiver can pass the call to emergency resources, at 416, and caregivers and relatives may be notified accordingly. When the caregiver determines that the monitored person is not in a severe state (e.g., there is no emergency), then, at 418, a caregiver may be notified to give help and love to the person and the call may be terminated. As in all cases, records of the conversation(s) and the actions taken may be recorded in the database. These records may be used for training the models for improved management of subsequent calls from the person being monitored. In one embodiment, the processor 30 may listen in and control training of machine-learning algorithms 480 used to implement the artificial intelligence models to generate (e.g., optimize) the models for managing calls.

At 420, when the decision indicates that there is an emergency (e.g., as indicated by a high score), then the processor may take the corresponding action indicated in Table 1. This may involve the processor 30 generating signals to cause the notification router to dispatch emergency services to the location of the person being monitored. In one embodiment, at 420, the conversation analyzer 35 may continue the conversation with the person until it is confirmed that the emergency resources have arrived, after which the call may be ended.

In addition to the foregoing operations, the processor 30 implementing the artificial intelligence algorithms of the models may monitor the dialog at all or pre-designated segments of the process follow in order to generate additional data for training the models. This data may cause the models to generate more accurate results, which, in turn, may improve the accuracy and effectiveness of the decisions rendered by the decision engine. This ultimately will inure to the benefit of the monitored person, by performing more informed and effective call management.

In one embodiment, the system may scale while using multiple events because of the learning algorithms implemented. The learning algorithms may allow the system to learn the responses of the person being monitored and more effectively analyze the conversations for purposes of generating an AI engine-driven response only.

### EXAMPLES

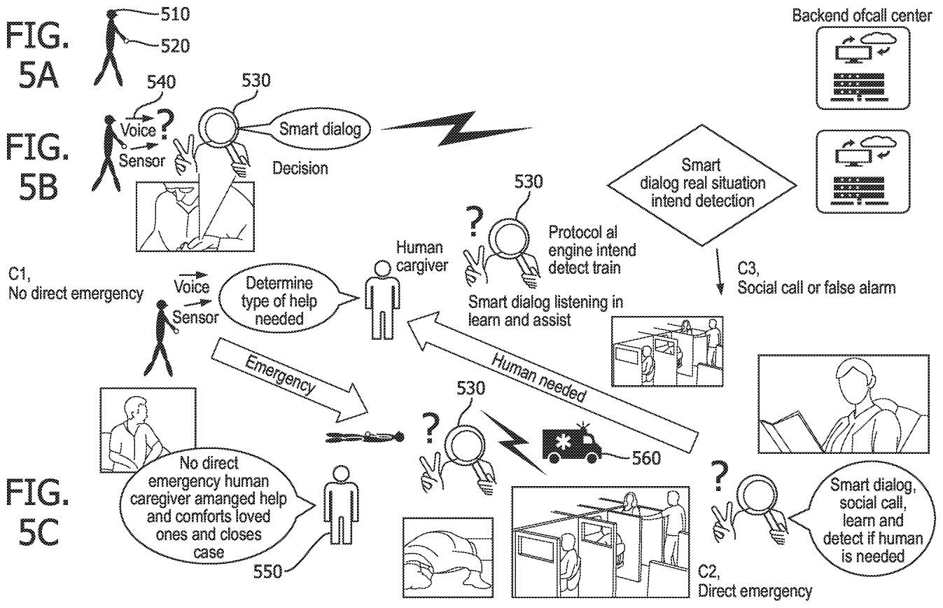

FIGS. 5A-5C illustrate an example of how the embodiments described herein may be applied in a practical application. The person to be monitored 510 user is wearing a smartwatch 520 that hosts an automatic fall detector (FIG. 5A.). When the detector detects a fall, the detector outputs an indication or notification to the user that the fall detector has been triggered and that an alarm signal will be sent to the call center system. The processor 30 of the call center system may then determine whether a cancel/revoke command is received from the user (FIG. 5B.). The predetermined time may be set an adjustable or fixed period of time in the application, and the cancel/revoke command may be generated, for example, when the user pushes a corresponding button or function on the smartwatch.

Irrespective of whether a cancel/revoke command is generated, the processor 30 activates the conversation analyzer to initiate and analyze a conversation using the virtual caregiver 540, for example, as previously described. The conversation may be referred to as a smart dialog conversation. During the conversation, voice signals 540 from the monitored person are received (through the smartwatch or another communication device) for analysis by the models of the conversation analyzer. These voice signals may or may not be accompanied by sensor data (providing additional information of the health of the person and/or circumstances relating to the event (e.g., alleged fall).

The decision engine, then, generates a decision based on the voice responses to make one or more of the determinations previously described. For example, the decision engine may determine if help is really needed (e.g., check if there an actual fall occurred, if a fall occurred as is being denied (e.g., false statements of “I am ok” when there is a high probability that a fall actually occurred and there is an emergency), if no fall occurred and the call is a social call, if the monitored person is in a confused state and additional assistance or questioning is to be performed). Additionally, or alternatively, the decision engine may render any of the other types of decisions previously discussed. A corresponding signal 550 may be sent to the back end of the call center (for a live operator) if certain decisions are rendered, for example, as indicated in Table 1 (e.g., based on a calculated score). In one embodiment, the signal 55 may be sent to a human caregiver (e.g., son or daughter, especially in non-emergency situations) instead of, or in addition to, the call center.

Referring to FIG. 5C, if the decision engine determines that it is unclear whether a fall has actually occurred and/or the patient is determined to be in a confused state (e.g., scenario C1—no direct emergency), then the processor 30 may perform any of the actions indicated in Table 1 (e.g., based on a computed score). In one embodiment, the processor 30 may activate an AI bot of the virtual caregiver 530 in order to obtain additional information. The conversation analyzer 35 may also listen to the voice responses in during the primary and/or AI bot interaction (e.g., between AI and human) (if implemented) to train the artificial intelligence models. Additionally, or alternatively, a human caregiver may be notified 550 for help and assistance.

If the decision engine determines that an actual fall has occurred and, for example, that the fall is being denied by the person being monitored (e.g., scenario C2—Direct Emergency), then the processor 30 may generate signals in to perform, for example, any of the actions indicated in Table 1 (e.g., based on a computed score). For example, the processor may generate signals to cause the notification router to dispatch emergency resources 560. Also, the processor 30 may hand-off the call to a live operator of the call center (through the notification router), who may provide personal assistance and stay on the call until it is confirmed that emergency resources have arrived. Also, the processor 30 may generate signals to notify a caregiver (e.g., relative) as indicated by the profile information stored for the person who fell. The processor 30 may also update the training of the modes of the conversation analyzer based on the voice responses and data, if available, received during the conversation.

If the decision engine determines that an actual fall has probably not occurred and/or the call is being made for social reasons (e.g., scenario C3—Social Call or False Alarm), then the processor 30 may perform any of the actions indicated in Table 1 (e.g., based on a computed score). The processor 30 may also update the training of the modes of the conversation analyzer based on the voice responses and data, if available, received during the conversation.

### Technological Innovations

In accordance with one or more of the aforementioned embodiments, a system and method is provided that makes use of Natural Language Processing models, conversation analysis, voice analysis, sensor data (e.g., through Philips PERS operator and 5* Why protocol), and sensor fusion combined with AI machine-learning Intent detection to manage alarms and/or other signals received from at least one sensor monitoring a person of interest. The sensor may be a fall detector or another type of sensor or monitoring device.

Upon the detection of a fall (or other possible emergency indicators), a call center system may not immediately contact emergency personnel or connect the call to a live operator in order to assist the person suspected of falling or who otherwise may require assistance. Instead, the system may initiate a conversation with the person using a virtual caregiver and then implement one or more models to analyze voice responses during the conversion. The system may wait a predetermined period of time (e.g., a time period for receiving a cancel/revoke signal) before initiating a call and conversation with the virtual caregiver.

The system may implement the virtual caregiver with a conversation analyzer that implements various models to assess the intent or emotion of the monitored person, voice inflection, context determination, text analysis, emotion detection, stress analysis, and/or various forms of natural language processing models to generate a decision concerning management of the call and what action, if any, to take in order to provide the most effective are to the potentially injured person, while at the same time preventing an unnecessary expenditure or allocation of emergency or call center resources. The conversation analyzer may also generate these decisions based on sensor data and profile information and may contact caregivers (e.g., relatives, friends, etc.) who may provide assistance or consolation. The services provided by the conversation analyzer and its attendant features are especially useful in assisting the elderly, who may be in denial of an actual fall, in a confused station, or who may be initiating alarms for social reasons where no fall actually occurred.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The processors, sensors, detectors, engines, conversation other analyzers, voice synthesizers, voice recognition and processing features, managers, artificial intelligence and other models and algorithms, routers, machine-learning and training logic, and other information generating, processing, and calculating features of the embodiments disclosed herein may be implemented in logic which, for example, may include hardware, software, or both. When implemented at least partially in hardware, processors, sensors, detectors, engines, conversation other analyzers, voice synthesizers, voice recognition and processing features, managers, artificial intelligence and other models and algorithms, routers, machine-learning and training logic, and other information generating, processing, and calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, processors, sensors, detectors, engines, conversation other analyzers, voice synthesizers, voice recognition and processing features, managers, artificial intelligence and other models and algorithms, routers, machine-learning and training logic, and other information generating, processing, and calculating features of the embodiments disclosed herein may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

We claim:

1. A system for managing a call, comprising:
a memory configured to store instructions; and
a processor configured to execute the instructions to implement a virtual caregiver to assist callers of a monitoring service, the virtual caregiver configured to receive sensor data from a monitoring device and activate a conversation analyzer in response to the sensor data, the conversation analyzer configured to:
initiate a call to a user of the monitoring device;
perform a conversation with the user during the call, the conversation performed in accordance with operations that include generating audible comments in a synthesized voice to interact with the user, the audible comments to elicit voice responses from the user containing information corresponding to the sensor data;
analyzing, using one or more models, audible features of the voice responses to interpret a condition of the user;
generating a decision based on the interpreted condition of the user; and
performing at least one action based on the decision.

2. The system of claim 1, wherein the audible features include one or more of voice inflection, pitch pattern, tone variations, volume fluctuations, or variable speech patterns.

3. The system of claim 2, wherein the condition is one of an intent, emotional state, or mental state of the user.

4. The system of claim 1, wherein:
the one or more models are artificial intelligence models that are to be trained based on patterns of audible features personalized to user, and
the processor configured to update training of the one or more models based on at least one of the voice responses, decision, or interpreted condition of the user.

5. The system of claim 1, wherein:
the monitoring device includes a fall detector, and
the sensor data indicates that the user has experienced a fall.

6. The system of claim 5, wherein the conversation analyzer is to generate a score based on output of the one or more models, the score indicative of a probability of the decision based on the interpreted condition.

7. The system of claim 5, wherein:
the interpreted condition includes an actual fall of the user;
the decision is the user is denying that the actual fall occurred; and
the at least one action includes generating signals to perform one or more of passing the call to a live operator, notifying an emergency resource for the user, or notifying a live caregiver.

8. The system of claim 5, wherein:
the interpreted condition includes that the user did not actually fall;
the decision is the user has pushed an alarm button on the fall detector in order to have a social call; and
the at least one action includes generating signals to perform one or more of providing options to the user or obtain additional information or notifying a live caregiver.

9. The system of claim 5, wherein:
the interpreted condition includes the user is in a confused state;
the decision is the user requires assistance; and
the at least one action includes one or more of notifying an emergency resource for the user or notifying a live caregiver.

10. The system of claim 1, wherein the conversation analyzer is configured to generate the decision based on the interpreted condition of the user and one or more of information included in the sensor data or profile information of the user.

11. A method for managing a call, comprising:
receiving sensor data from a monitoring device; and
activating a conversation analyzer in response to the sensor data, wherein activating the conversation analyzer includes:
initiating a call to a user of the monitoring device;
performing a conversation with the user during the call, said performing including generating audible comments in a synthesized voice to interact with the user, the audible comments eliciting voice responses from the user containing information corresponding to the sensor data;
analyzing, using one or more models, audible features of the voice responses to interpret a condition of the user;
generating a decision based on the interpreted condition of the user; and
performing at least one action based on the decision.

12. The method of claim 11, wherein the audible features include one or more of voice inflection, pitch pattern, tone variations, volume fluctuations, or variable speech patterns.

13. The method of claim 12, wherein the condition is one of an intent, emotional state, or mental state of the user.

14. The method of claim 11, wherein:
the one or more models are artificial intelligence models that are trained based on patterns of audible features personalized to user, and
the method includes updating training of the one or more models based on at least one of the voice responses, decision, or interpreted condition of the user.

15. The method of claim 11, wherein:
the monitoring device includes a fall detector, and
the sensor data indicates that the user has experienced a fall.

16. The method of claim 15, further comprising:
generating a score based on output of the one or more models,
wherein the score is indicative of a probability of the decision based on the interpreted condition.

17. The method of claim 15, wherein:
the interpreted condition includes an actual fall of the user;
the decision is the user is denying that the actual fall occurred; and
the at least one action includes generating signals to perform one or more of passing the call to a live operator, notifying an emergency resource for the user, or notifying a live caregiver.

18. The method of claim 15, wherein:
the interpreted condition includes that the user did not actually fall;
the decision is the user has pushed an alarm button on the fall detector in order to have a social call; and
the at least one action includes generating signals to perform one or more of providing options to the user or obtain additional information or notifying a live caregiver.

19. The method of claim 15, wherein:
the interpreted condition includes the user is in a confused state;
the decision is the user requires assistance; and
the at least one action includes one or more of notifying an emergency resource for the user or notifying a live caregiver.

20. The method of claim 11, wherein generating the decision is performed based on the interpreted condition of the user and one or more of information included in the sensor data or profile information of the user.
